(19) United States
(12) Patent Application Publication
(10) Pub. No.: US 2026/0335276 A1
(43) Pub. Date: Oct. 29, 2026

(54) MODULATING CHONDROCYTE HYPERTROPHY SIGNALLING PATHWAYS FOR OSTEOARTHRITIS TREATMENT

(71) Applicant: Center for Neuromusculoskeletal Restorative Medicine Limited, Pak Shek Kok (HK)

(72) Inventors: Chao WAN, Shatin (HK); Larry Yung-tim LEUNG, Shatin (HK); Fengjie ZHANG, Shatin (HK)

(21) Appl. No.: 19/449,776

(22) Filed: Jan. 15, 2026

Related U.S. Application Data
(60) Provisional application No. 63/796,151, filed on Apr. 28, 2025.

(51) Int. Cl. A61K 31/357; A61K 9/00; A61K 45/06; A61P 19/02

(52) U.S. Cl. CPC A61K 31/357; A61K 9/0019; A61K 45/06; A61P 19/02

(57) ABSTRACT

A pharmaceutical composition for treating osteoarthritis comprising an effective amount of a small molecule inhibitor and its pharmaceutically acceptable carrier. Also disclosed is a method for treating osteoarthritis (OA) comprising administering an effective amount of a small molecule inhibitor that modulates a signalling pathway associated with chondrocyte hypertrophy to a subject in need.

### CROSS-REFERENCE TO RELATED APPLICATIONS

The present application claims priority to U.S. Provisional Application No. 63/796,151, filed on Apr. 28, 2025.

### FIELD OF THE INVENTION

The present invention relates to a pharmaceutical composition for treating osteoarthritis by administering an effective amount of a small molecule inhibitor that modulates a signalling pathway associated with chondrocyte hypertrophy to a subject in need.

### BACKGROUND OF THE INVENTION

Degenerative joint diseases (DJDs) are a class of progressive musculoskeletal disorders that are characterized by the gradual deterioration of cartilage, leading to joint pain, stiffness, and impaired mobility. Of all DJDs, osteoarthritis is the most common, affecting millions of people worldwide, while other DJDs are, in turn, post-traumatic arthritis, osteochondritis dissecans, and degenerative disc disease. These conditions are driven by factors such as aging, mechanical stress, inflammation, and cellular dysregulation, ultimately resulting in cartilage loss and joint dysfunction.

Articular cartilage injury and degeneration or osteoarthritis (OA) of the knee occur frequently and represent a major health problem worldwide. OA is a degenerative joint disease that causes pain, stiffness, and reduces mobility. Due to the avascular and aneural nature of the articular cartilage, it has a very limited regenerative potential. Despite their high prevalence, there is currently no FDA-approved disease-modifying treatment for DJDs, highlighting the urgent need for innovative therapeutic strategies.

As the existing clinical techniques in managing OA mainly focus on relieving symptoms, the current medical treatment methods cannot address the underlying cartilage degeneration, as they would not arrest, slow or reverse progression of OA-related structural damage in the joint. Therefore, effective methods in treating OA are still lacking.

A key challenge in treating OA is finding a cost-effective and patient-friendly alternative to joint replacement surgery. Surgical interventions come with high healthcare costs, significant post-operative care, and invasive procedures that can be burdensome for patients. There is a need for a minimally invasive approach that reduces these challenges while maintaining the effectiveness of treatments. The application of small molecule inhibitors is found to be more cost-effective for treating OA.

For example, US Patent Application No. 2021369714 A1 disclosed an inhibitor that targets IGF-IR signalling to a joint to treat or prevent OA, therefore, inhibits phosphorylation of the IGF-1R. By employing a small molecule tyrosine kinase inhibitor, namely, NVP-AEW541 (among others), the inhibitor is surgically placed into the joint of the subject in a form of scaffold such as nanorods or microrods. It is said that the small molecule inhibitor in this invention prevents or decreases abnormal chondrocyte proliferation, abnormal vasculature formation, cartilage degradation, articular damage and/or osteophyte formation associated with development and/or progression of OA. However, this invention has not disclosed the reduction of any hypertrophic markers such Runx2, ALP, ColX, IHH, VEGF that eventually prevent proteoglycan and collagen type II loss, restores ICRS scores, and directly contributes to cartilage repair in OA-related conditions.

Additionally, U.S. Pat. No. 9,855,266 B2 disclosed a method for treating OA by administering a compound through intra-articular injection to an osteoarthritic joint in the subject as a single-dose treatment. The target is Mouse Double Minute 2 homolog (MDM2). Said compound disclosed in this invention is a small molecule senolytic agent that selectively kills senescent cells over non-senescent cells. A senolytic agent is also added, the agent namely, 4-[[(4S,5R)-4,5-bis(4-chlorophenyl)-4,5-dihydro-2-[4-methoxy-2-(1-methylethoxy)phenyl]-1H-imidazol-1-yl]carbonyl]-2-piperazinone or PP242. The treatment using the senolytic agent results in inhibition of erosion of a proteoglycan layer in the osteoarthritic joint and promoting production of collagen Type II, thus reducing or inhibiting of loss or erosion of proteoglycan layers in a joint, reduces inflammation in the affected joint by promoting production of collagen (e.g., type 2 collagen). However, in OA, inhibiting MDM2 might trigger unwanted apoptosis in chondrocytes, leading to faster cartilage loss rather than repair, as it often enhances p53 activity to promote cell death in tumors.

In addition to the above, International Patent Publication No. WO 2008067427 A2 relates to the use of insulin-like growth factor receptor antagonists for weight maintenance, weight reduction, and treatment of obesity. The antagonists in this invention target IGF-1R and are selected from various groups of suitable compounds such as small molecules, proteins, polypeptides, IGF mimetics, antisense oligodeoxynucleotides, antisense RNAs, small inhibitory RNAs, triple helix forming nucleic acids, dominant negative mutants, and soluble receptor expression. However, the IGF-1R antagonists may have systemic metabolic effects, potentially affecting muscle mass, glucose metabolism, and overall IGF signalling.

### SUMMARY OF THE INVENTION

It is an objective of the present invention to provide a method for treating the progression of degenerative joint diseases such as OA by controlling the pathological process of abnormal chondrocyte hypertrophy.

It is also an objective of the present invention to provide an FDA-approvable therapeutic option for treating the progression of degenerative joint diseases such as OA. More specifically, to provide a potential disease-modifying osteoarthritis drug (DMOAD) using Insulin-like Growth Factor 1 Receptor (IGF-1R) small molecule inhibitor.

Accordingly, these objectives can be achieved by following the teachings of the present invention, which relates to a pharmaceutical composition for treating OA comprising an effective amount of a small molecule inhibitor and its pharmaceutically acceptable carrier.

These objectives can also be achieved by following the teachings of the present invention, which relates to a method for treating OA comprising administering an effective amount of a small molecule inhibitor that modulates a signalling pathway associated with chondrocyte hypertrophy to a subject in need.

### BRIEF DESCRIPTION OF DRAWINGS

The features of the invention will be more readily understood and appreciated from the following detailed description when read in conjunction with the accompanying drawings of the preferred embodiment of the present invention.

FIG. 1 illustrates a method of administration of a pharmaceutical composition in preventing chondrocyte hypertrophy in an osteoarthritic joint in vitro and in vivo.

FIGS. 2A and 2B illustrate the ability of defined doses of Picropodophyllin (PPP) in maintaining chondrocyte proliferation and viability for three and seven days, respectively.

FIGS. 3A and 3B illustrate the ability of defined doses of PPP in maintaining the proteoglycan synthesis in the in vitro micro-mass culture.

FIG. 4 illustrates the ability of defined dose of PPP in reducing hypertrophic marker gene expression of the mice chondrocytes in inflammatory condition after PPP treatment.

FIG. 5A illustrates the Safranin O/Fast green histological staining for visualizing proteoglycans (red) in mouse articular cartilage.

FIG. 5B illustrates the quantification of the relative integral optical density (IOD) of Safranin O staining.

FIG. 5C illustrates the quantification of the ICRS II scores of the articular cartilage layer.

FIGS. 6A-D illustrate that PPP prevents degeneration of the OA joint by preserving collagen.

FIGS. 7A-C illustrate that PPP prevents articular cartilage chondrocytes from undergoing hypertrophy in the mouse OA model.

FIGS. 8A-D illustrate that PPP inhibits hypertrophy makers expression in the articular cartilage of the mice OA model.

FIG. 9 illustrates that intra-articular injection of PPP does not induce significant toxicity to the kidney.

FIGS. 10A-B illustrate that intra-articular injection of PPP does not induce significant toxicity to the liver.

### DETAILED DESCRIPTION OF THE INVENTION

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

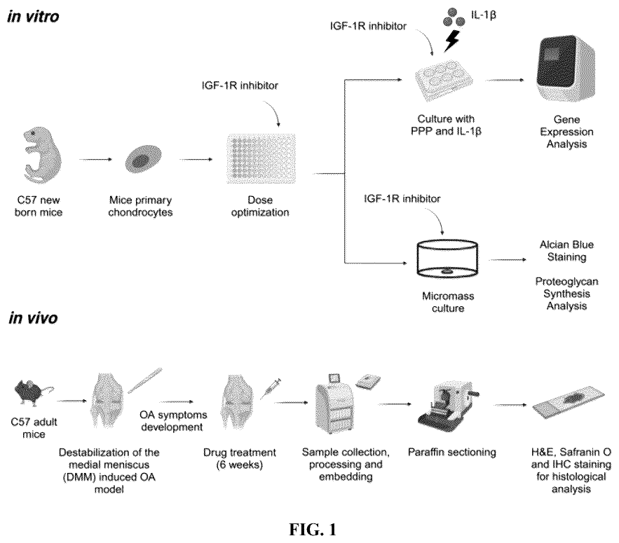

The present invention teaches a pharmaceutical composition for treating OA comprising an effective amount of a small molecule inhibitor and its pharmaceutically acceptable carrier. This is illustrated in FIG. 1.

In an embodiment of the present invention, the small molecule inhibitor is an Insulin-like Growth Factor 1 Receptor (IGF-1R) inhibitor that inhibits IGF-1R signal pathway.

In an embodiment of the present invention, the effective amount of the small molecule inhibitor is between 50 nM to 100 nM.

The present invention also teaches a method for treating OA comprising administering an effective amount of a small molecule inhibitor that modulates a signalling pathway associated with chondrocyte hypertrophy to a subject in need.

In an embodiment of the present invention, the administration of the effective amount of the small molecule inhibitor is administered intra-articularly at an affected joint of the subject in need.

In an embodiment of the present invention, the method further comprising administering the effective amount of the small molecule inhibitor with an anti-inflammatory agent.

The present invention also teaches a method for preventing chondrocyte hypertrophy in an osteoarthritic joint of a subject, comprising administering an effective amount of a small molecule inhibitor intra-articularly to the joint, wherein the administration reduces hypertrophic differentiation of chondrocytes and alleviates OA-associated cartilage degeneration.

In an embodiment of the present invention, the reduction in chondrocyte hypertrophy leads to decreased expression of hypertrophic markers, including but not limited to Runt-related transcription factor 2 (Runx2), Alkaline phosphatase (ALP), Collagen type X (Col X), Indian Hedgehog (IHH), Vascular Endothelial Growth Factor (VEGF), Matrix Metalloproteinase 13 (MMP13).

In an embodiment of the present invention, the administration of the small molecule inhibitor maintains chondrocyte viability, proliferation, and proteoglycan synthesis.

The present invention also teaches a use of a small molecule inhibitor in the manufacture of a medicament for the treatment of OA by preventing or reducing chondrocyte hypertrophy.

In one embodiment, the present invention provides an approach that utilizes small molecule inhibitor of IGF-1R to suppress chondrocyte hypertrophy and maintain chondrocyte phenotype for cartilage repair in the OA-related conditions. This invention aims to control abnormal chondrocyte hypertrophy, which is a pathological process during the progression of OA. This process is characterized by the aberrant enlargement and terminal differentiation of the chondrocytes, eventually leading to remodelling and mineralization of the existing hyaline cartilage.

More specifically, the use of the small molecule inhibitor of IGF-IR is administered via defined doses of a potent small molecule therapeutic agent IGF-1R inhibitor including but not limited to PPP to reduce chondrocyte hypertrophy and alleviate the degeneration of the cartilage in the OA condition.

The existing methods in managing OA are mainly conservative treatments, so the current approach in treating OA can bridge the gap between the treatment of early and late-stage OA conditions and offer alternative treatment methods for patients with OA. More specifically, one of the administration techniques, the intra-articular injection of the small molecule does not require surgery, and it only requires minimal invasive procedures, so it is a more patient-friendly approach than joint replacement surgeries. This method can reduce healthcare costs relating to surgeries and post-operative care.

More specifically, the current invention is a method for treating OA by controlling chondrocyte hypertrophy through the application of an IGF-1R inhibitor (PPP) to modulate IGF-1R signalling in chondrocytes.

### Examples

Experiments are conducted to investigate the effect of IGF-1R inhibition on controlling chondrocyte hypertrophy in OA conditions.

In the in vitro experiments, primary chondrocytes are isolated from newborn mice, and the doses of IGF-1R inhibitor (PPP) are optimized with cell viability assay. The chondrocytes are treated with defined doses of PPP for gene expression and proteoglycan synthesis analysis. In the in vivo experiments, destabilization of the medial meniscus surgery is conducted in the C57BL/6 adult mice to induce OA. The mice receive intra-articular injection of the PPP for 6 weeks, followed by knee joint sample collection, processing, paraffin embedding and sectioning. The histological sections of the knee joint are stained with H&E, Safranin O and IHC for articular cartilage markers (Scheme 1). Defined doses of small molecule inhibitor of IGF-1R can maintain chondrocyte proliferation, viability, and maintain the proteoglycan synthesis in the in vitro chondrocyte micro-mass culture. This molecule reduces the hypertrophic marker gene expression of chondrocytes following treatment under inflammatory conditions (IL-1β treatment). This molecule prevents the loss of proteoglycan, and collagen type II (COL II), restores the ICRS II scores of the articular cartilage, and downregulates chondrocyte hypertrophy makers (e.g., MMP13 and COL X) in the destabilization of medial meniscus induced mouse OA model.

### Preliminary Experimental Results

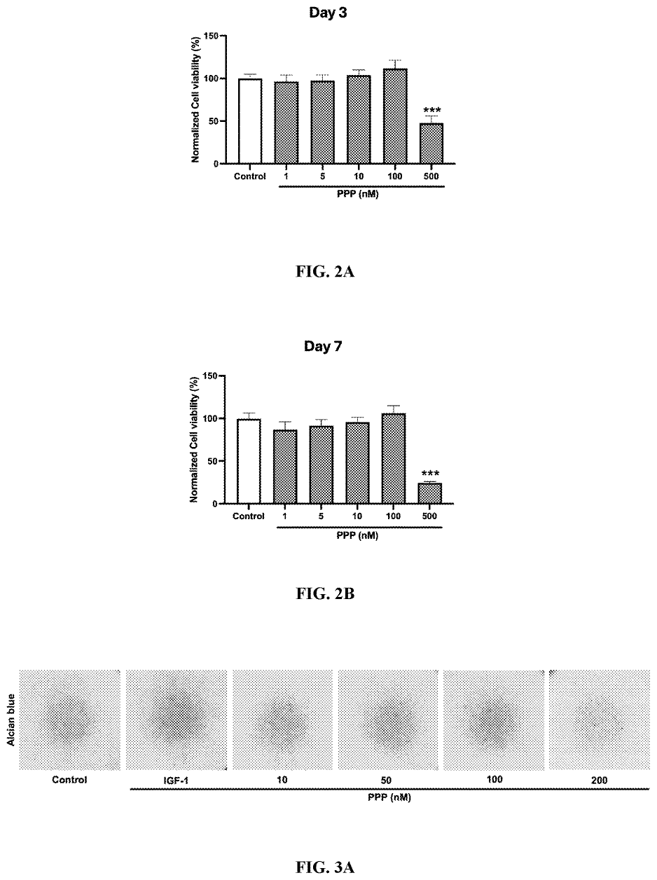

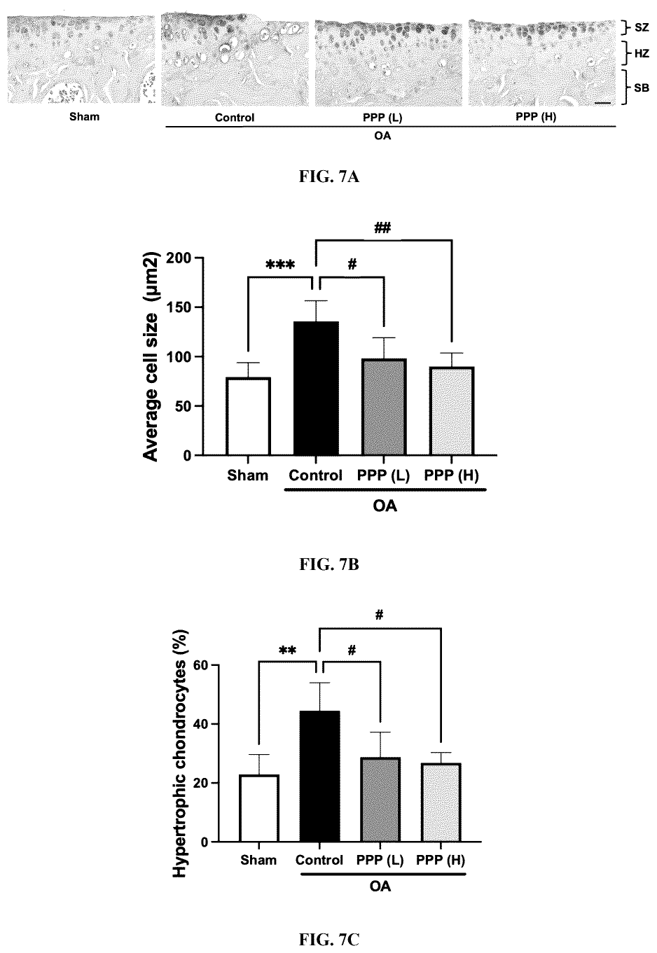

As illustrated in FIGS. 2A and 2B, the primary mice chondrocytes are cultured in different dosages of the IGF-1R inhibitor (PPP) for 3 (FIG. 2A) and 7 days (FIG. 2B). Cell proliferation assay is conducted to test the optimized doses of the IGF-1R inhibitor on mice chondrocytes. Data is analyzed by One-way ANOVA, ***P<0.001, compared with control, n=4.

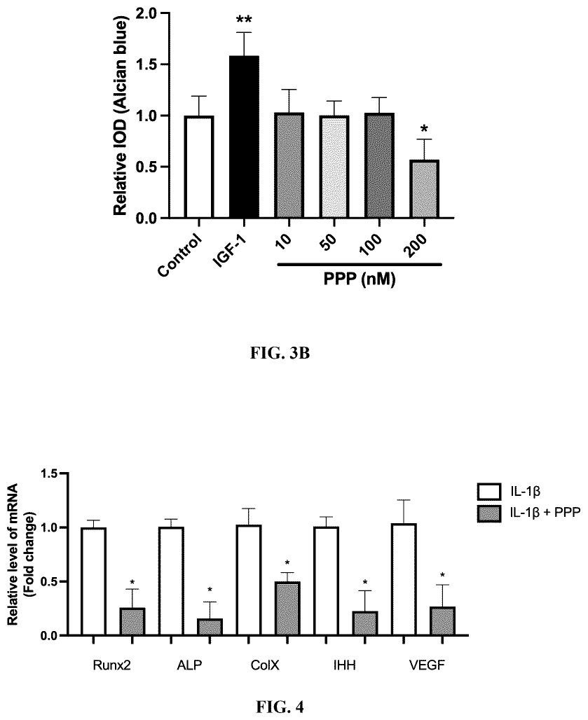

FIG. 3A illustrates mice chondrocytes that are treated with 20 nM of IGF-1 or different dosages of PPP for 7 days and stained for Alcian blue for visualizing the proteoglycan synthesized by chondrocytes, whereas FIG. 3B illustrates quantification of the relative integrated optical density (IOD) of Alcian blue staining in different groups. Data is analyzed by One-way ANOVA, *P<0.05, compared with control, n=3.

FIG. 4 illustrates mice chondrocytes are seeded into the 6 well plate and pre-treated in 10 ng/mL of IL-1β for 24 hours before the treatment. 100 nM of PPP is added to the culture and incubated for 30 mins before harvesting for gene expression analysis. The mRNA expression level is shown as the ratio of the target gene to GAPDH. Data is analyzed by Student t-test, *P<0.05, compared with control, n=3.

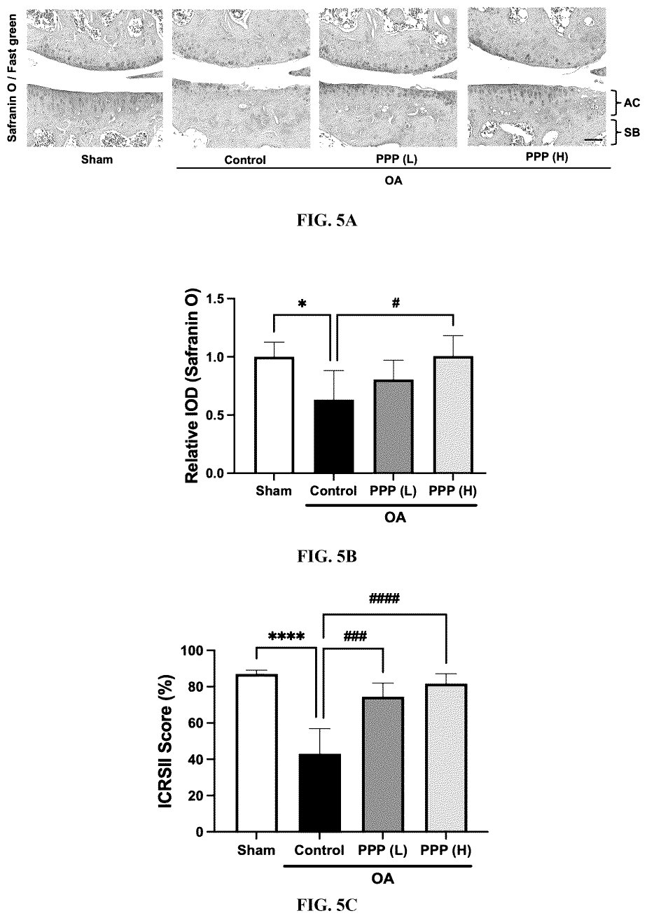

FIG. 5A, Safranin O/Fast green histological staining is used for visualizing proteoglycans (red) in mouse articular cartilage following 6 weeks of saline (control group) or PPP (treatment group) intra-articular administration. FIG. 5B further illustrates that the quantification of the relative IOD of Safranin O staining. FIG. 5C illustrates the quantification of the ICRS II scores of the articular cartilage layer. L, low dose (50 nM); H, high dose (100 nM); AC, articular cartilage; SB, subchondral bone. Scale bar=100 μm. *P<0.05, ****P≤0.0001 compared with sham group. Data is analyzed by One-way ANOVA, #P<0.05, ###P<0.001, ####P≤0.0001 compared with DMM control group, n=5.

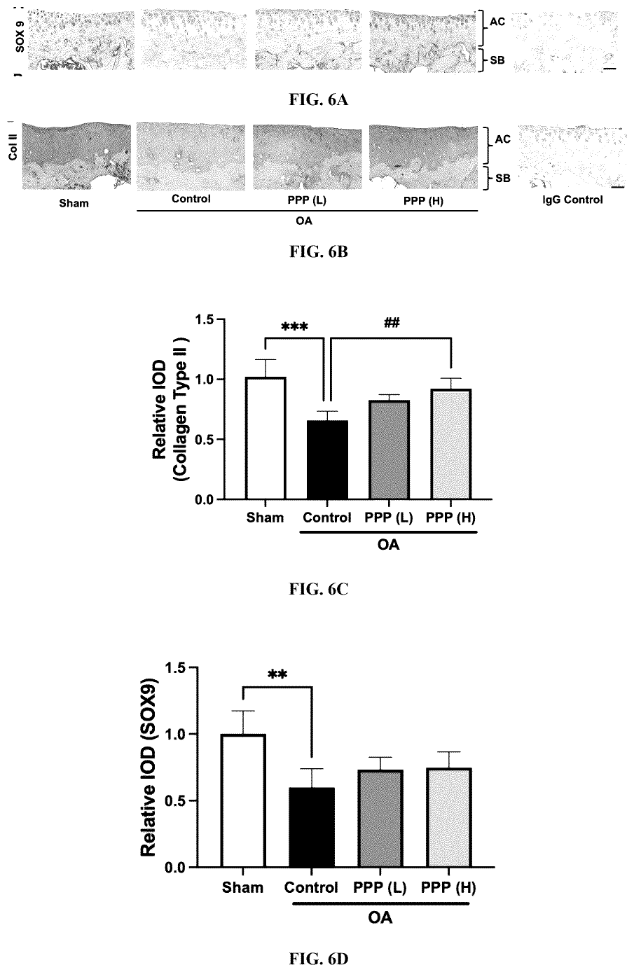

FIGS. 6A-D illustrate that PPP prevents the degeneration of the OA joint by preserving collagen. More specifically, FIG. 6A & FIG. 6B revealed in Immunohistochemistry (IHC) staining of SOX9 and Col II in mouse articular cartilage following 6 weeks of saline (control group) or PPP (treatment group) intra-articular administration. FIG. 6C & FIG. 6D revealed the IOD quantification of SOX9 and Col II in the articular cartilage respectively. L, low dose (50 nM); H, high dose (100 nM); AC, articular cartilage; SB, subchondral bone. Scale bar=100 μm. **P<0.01, ***P<0.001 compared with sham control. Data is analyzed by One-way ANOVA, ##P<0.01 compared with DMM control group, n=5.

FIGS. 7A-C illustrate that PPP prevents articular cartilage chondrocytes from undergoing hypertrophy in the mouse OA model. For example, FIG. 7A shows histological staining of Hematoxylin and Eosin (H&E) for visualizing chondrocytes in mouse articular cartilage following 6 weeks of saline (control group) or PPP (treatment group) intra-articular administration. FIG. 7B shows the quantification of the average chondrocytes size in the articular cartilage. L, low dose (50 nM); H, high dose (100 nM); SZ, superficial zone; HZ, hypertrophic zone; SB, subchondral bone. Scale bar=100 μm. *P<0.05, **P<0.01, ***P<0.001 compared to sham control. Data is analyzed by One-way ANOVA, #P<0.05, ##P<0.01 compared with DMM control group, n=5. FIG. 7C shows the quantification of the percentage of hypertrophic chondrocytes in the articular cartilage. L, low dose (50 nM); H, high dose (100 nM). **P<0.01, compared to sham control. #P<0.05, compared with DMM control group, n=5.

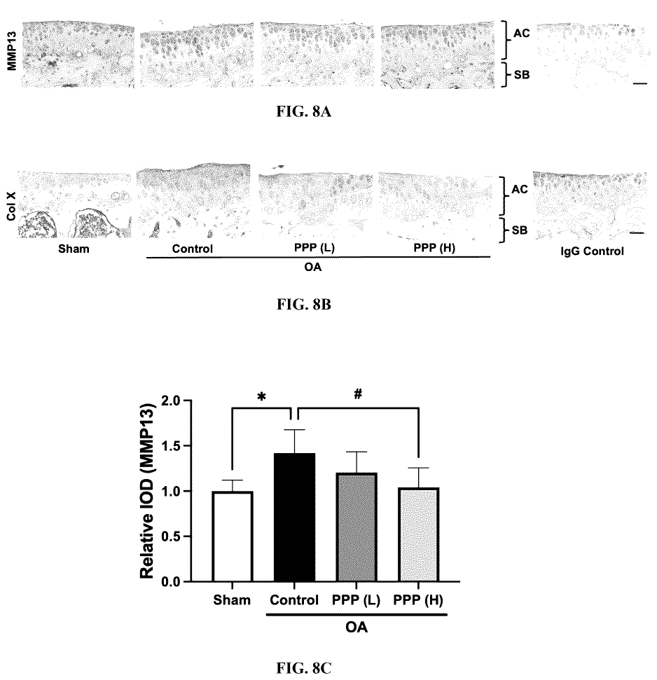

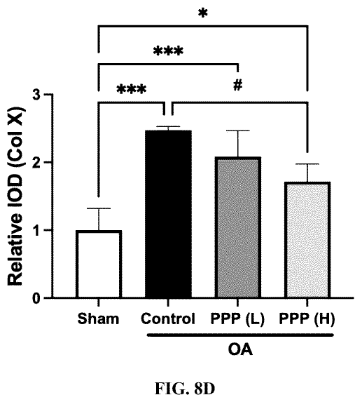

FIGS. 8A-D illustrate that PPP inhibits hypertrophy makers expression in the articular cartilage of the mice OA model. More specifically, FIGS. 8A-B shows immunohistochemistry (IHC) staining of Col X and MMP13 in mouse articular cartilage following 6 weeks of saline (control group) or PPP (treatment group) intra-articular administration. FIGS. 8C-D shows that the IOD quantification of Col X and MMP13 in the articular cartilage respectively. L, low dose (50 nM); H, high dose (100 nM); AC, articular cartilage; SB, subchondral bone. Scale bar=100 μm. Data is analyzed by One-way ANOVA, *P<0.05, ***P<0.001 compared with sham control. ##P<0.05 compared with DMM control group, n=5.

FIG. 9 illustrates that intra-articular injection of PPP does not induce significant toxicity to the kidney. More specifically, it shows that H&E staining for visualizing the structures of the kidney following 6 weeks of saline (control group) or PPP (treatment group) intra-articular administration. L, low dose (50 nM); H, high dose (100 nM). Scale bar=200 μm.

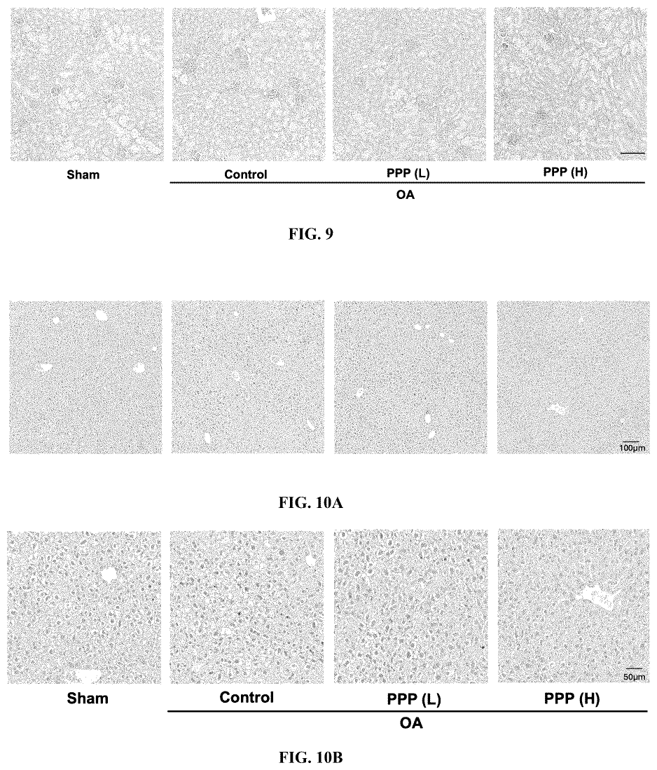

FIGS. 10A-B illustrate that the intra-articular injection of PPP does not induce significant toxicity to the liver. More specifically, the H&E staining for visualizing the structures of the liver of different treatment groups at 7 weeks post-surgery. The structures are captured in high and low magnification respectively. L, low dose (50 nM); H, high dose (100 nM). Scale bar=100 and 50 μm for figure A and B respectively.

The present invention explained above is not limited to the aforementioned embodiment and drawings, and it will be obvious to those having an ordinary skill in the art of the present invention that various replacements, deformations, and changes may be made without departing from the scope of the invention.

1. A pharmaceutical composition for treating osteoarthritis comprising an effective amount of a small molecule inhibitor and its pharmaceutically acceptable carrier.

2. The pharmaceutical composition of claim 1, wherein the small molecule inhibitor is an Insulin-like Growth Factor 1 Receptor (IGF-1R) inhibitor that inhibits IGF-1R signal pathway.

3. The pharmaceutical composition of claim 1, wherein the effective amount of the small molecule inhibitor is between 50 nM to 100 nM.

4. A method for treating osteoarthritis (OA) comprising administering an effective amount of a small molecule inhibitor that modulates a signalling pathway associated with chondrocyte hypertrophy to a subject in need.

5. The method of claim 4, wherein the administration of the effective amount of the small molecule inhibitor is administered intra-articularly at an affected joint of the subject in need.

6. The method of claim 4, wherein the method further comprising administering the effective amount of the small molecule inhibitor with an anti-inflammatory agent.

7. A method for preventing chondrocyte hypertrophy in an osteoarthritic joint of a subject, comprising administering an effective amount of a small molecule inhibitor intra-articularly to the joint, wherein the administration reduces hypertrophic differentiation of chondrocytes and alleviates OA-associated cartilage degeneration.

8. The method of claim 7, wherein the reduction in chondrocyte hypertrophy leads to decreased expression of hypertrophic markers, including but not limited to Runt-related transcription factor 2 (Runx2), Alkaline phosphatase (ALP), Collagen type X (Col X), Indian Hedgehog (IHH), Vascular Endothelial Growth Factor (VEGF), Matrix Metalloproteinase 13 (MMP13).

9. The method of claim 7, wherein the administration of the small molecule inhibitor maintains chondrocyte viability, proliferation, and proteoglycan synthesis.

10. Use of a small molecule inhibitor in the manufacture of a medicament for the treatment of osteoarthritis by preventing or reducing chondrocyte hypertrophy.
